(19) United States
(12) Patent Application Publication
(10) Pub. No.: US 2021/0118556 A1
(43) Pub. Date: Apr. 22, 2021

(54) SYSTEMS AND METHODS FOR DYNAMIC GENERATION OF STRUCTURED QUALITY INDICATORS AND MANAGEMENT THEREOF

(71) Applicant: KONINKLIJKE PHILIPS N.V., ElNDHOVEN (NL)

(72) Inventors: Jennifer CAFFAREL, EINDHOVEN (NL); Muhammad ASIM, EINDHOVEN (NL); Claudia FISCHER, CAMBRIDGE, MA (US); Jarno Mikael RIISTAMA, WAALRE (NL); Gijs GELEIJNSE, GELDROP (NL); Francisco ROEDER, SANTA CATARINA (BR)

(21) Appl. No.: 17/041,381

(22) PCT Filed: Mar. 28, 2019
(86) PCT No.: PCT/EP2019/057962

§ 371 Date: Sep. 24, 2020

Related U.S. Application Data
(60) Provisional application No. 62/650,394, filed on Mar. 30, 2018.

(51) Int. Cl. G16H 40/20; G06F 16/28; G06F 16/2455; G06F 16/2452; G06Q 10/06

(52) U.S. Cl. CPC G16H 40/20; G06F 16/285; G06Q 10/06395; G06F 16/24522; G06F 16/2455

(57) ABSTRACT

Systems and methods are provided for healthcare quality measurement. A user interaction module receives a first unstructured quality indicator from a first healthcare quality delivery system. A natural language processing engine, including a quality indicator framework and a library parses the unstructured quality indicator to identify key words and relationships therebetween. A structured quality indicator generator generates suggested structured quality indicators from unstructured quality indicators. The suggested structured quality indicators are standardized according to a predetermined standard A quality measure engine generates a query corresponding to a selected one of the structured quality indicators. A data repository outputs data from executing the query thereon. A quality measure dashboard outputs the result data.

### FIELD

The present application generally relates to quality indicators. More particularly, the present application relates to systems and methods for measuring healthcare quality by dynamically specifying quality indicators and providing corresponding structured quality indicators.

### BACKGROUND

Healthcare delivery entities are hospitals, institutions and/or individual practitioners that provide healthcare services to individuals. In recent years, there has been an increased focus on monitoring and improving the delivery of healthcare around the globe. Traditionally, healthcare delivery has been driven by volume, meaning that healthcare delivery entities are motivated to increase or maximize the volume of healthcare services, visits, hospitalizations and tests that they provide.

More recently, there is a growing trend in which healthcare delivery is shifting from being volume driven to being outcome or value driven. This means that healthcare delivery entities are being incentivized to provide high quality healthcare while minimizing costs, rather than simply providing the maximum volume of healthcare. One way in which healthcare delivery entities are being incentivized is by the implementation of payment systems in which healthcare delivery entities (e.g., Accountable Care Organizations (ACOs)) are paid using a pay-for-performance model.

This shift to outcome or value driven service has thus increased the importance of defining, monitoring and measuring the quality of healthcare, namely focusing on safe, effective, patient-centered, timely, efficient and equitable healthcare delivery. Healthcare quality measurements are used by emerging outcome or value driven payment models, for example, to benchmark performance against other providers, thereby improving transparency, accountability and quality; reward or penalize healthcare delivery entities or services that either meet or do not meet certain quality criteria; or conform to medical, environmental and other like standards or guidelines related to healthcare delivery.

Measuring and monitoring the quality of healthcare is therefore an important component in the business of healthcare delivery entities. Members, staff, directors and officers (e.g., chief financial officers (CFOs), chief executive officers (CEOs)) of healthcare delivery entities are tasked with measuring and monitoring the quality of healthcare provided at their respective entities. Healthcare delivery entities are faced with efficiently, accurately and dynamically obtaining health quality measurements, all while dealing with the challenges of a dynamic healthcare environment, such as fluctuating supply costs, quality requirements, changing patient volumes, staffing shortages, and the like.

Healthcare quality is measured using quality measures or indicators, which may also be referred to as key performance indicators (KPIs). Quality indicators are often developed or endorsed by organizations such as the National Quality Forum (NQF). The quality indicators are quantitative tools that are used to assess the clinical efficacy and performance of a healthcare delivery entity or individual. The efficacy and performance is quantified in relation to a specified action, process or outcome of clinical care. Quality indicators are typically developed based on well-defined clinical guidelines and evidence such as outcomes of research and clinical trials. Quality indicators are also designed to determine whether appropriate care has been provided given a set of clinical criteria and an evidence base.

However, healthcare quality is traditionally being measured using healthcare quality indicators that are static, complex, inflexible and inefficient. For instance, current ways of measuring and obtaining healthcare quality requires an end-user, such as a C-level member or data analyst of a healthcare delivery entity, to select a static quality indicator for execution from a predetermined and fixed list or set of quality indicators.

While other ways of measuring healthcare quality employ more flexible healthcare quality indicators, such approaches are inefficient and complex. For instance, current ways of developing and executing healthcare quality indicators do not allow end users to rapidly seek answers to thousands of questions related to quality improvement, in a manner that allows a continuous insight into how care is provided to their respective population. Moreover, to generate a custom quality indicator, an end user must provide the relevant parts of the desired quality indicator. So that the quality indicator is properly executable against a database, the quality indicator (and/or a corresponding query) must be generated with an understanding of how the data in the database is structured. This information is generally known by architects and developers of a system or database. However, C-level members of the healthcare delivery entity or other like end users may not have access to the framework of the data or likely may not be able to understand how to apply that information to generate the desired quality indicator.

There is a need therefore for improved systems and methods that enable measuring healthcare quality using flexible and dynamic quality indicators that are executable against different data sets. There is also a need for the quality indicators to be intuitively and efficiently generated, executed and visualized according to the needs of the end user, while allowing, for example, those quality indicators to able to be aggregated, presented on either or both a treatment level and/or a patient level, and to have different exclusion criteria applied. There is also a need for the endorsed quality indicators to be created based on an end user's unstructured input.

### SUMMARY

The present application provides systems and methods for measuring healthcare quality by dynamically specifying quality indicators and generating structured quality indicators

In some example embodiments, a healthcare quality measurement system comprises at least one memory operable to store a data repository, a first database and a second database; a processor communicatively coupled to the at least one memory. The processor is operable to: receive an unstructured quality indicator from one of a plurality of end-user systems; parse the unstructured quality indicator to identify key words, and relationships therebetween; map the key words to categories of a quality indicator framework, the categories of the quality indicator framework corresponding to one or more constituent parts of one or more candidate structured quality indicators; identify one or more suggested structured quality indicators from among the one or more candidate structured quality indicators, based at least on the key words mapped to the categories of the quality indicator framework; receive a selection of a structured quality indicator from among the one or more suggested structured quality indicators; generate a query corresponding to the structured quality indicator, the query being generated in a query language executable on the data repository; execute the query against the data repository to obtain result data, the results including information relating to the unstructured quality indicator and obtained based on the structured quality indicator; and output the result data obtained by executing the query, wherein the candidate structured quality indicators are quality indicators standardized according to a predetermined standard.

In some example embodiments, the result data is output via a quality measurement dashboard.

In some example embodiments, the predetermined standard is Health Quality Measures Format (HQMF) or Health Level Seven (HL7).

In some example embodiments, the categories of the quality indicator framework include required categories and optional categories, and wherein the processor is operable to identify the one or more suggested structured quality indicators upon mapping at least a portion of the key words to the required categories of the quality indicator framework.

In some example embodiments, the query language is SQL or JavaScript.

In some example embodiments, the first database and the second database are linked, wherein the first database stores the selected structured quality indicator, and wherein the second database stores the unstructured quality indicator in association with the corresponding selected structured quality indicator.

In some example embodiments, the parsing of the unstructured quality indicator to identify key words includes identifying, using a library comprising one or more dictionaries, synonyms or corresponding official terminology for one or more words in the unstructured quality indicator.

In some example embodiments, a method of providing healthcare quality measurements comprises: receiving an unstructured quality indicator from one of a plurality of end-user system; parsing the unstructured quality indicator to identify key words, and relationships therebetween; mapping the key words to categories of a quality indicator framework, the categories of the quality indicator framework corresponding to one or more constituent parts of one or more candidate structured quality indicators; identifying one or more suggested structured quality indicators from among the one or more candidate quality indicators, based at least on the key words mapped to the categories of the quality indicator framework; receiving a selection of a structured quality indicator from among the one or more suggested structured quality indicators; generating a query corresponding to the structured quality indicator, the query being generated in a query language executable on a data repository; executing the query against the data repository to obtain result data, the results including information relating to the unstructured quality indicator and obtained based on the structured quality indicator; and outputting the result data obtained by executing the query, wherein the candidate structured quality indicators are quality indicators standardized according to a predetermined standard.

In some example embodiments, the result data is output via a quality measurement dashboard.

In some example embodiments, the predetermined standard is Health Quality Measures Format (HQMF) or Health Level Seven (HL7).

In some example embodiments, the categories of the quality indicator framework include required categories and optional categories, and wherein the method further comprises identifying the one or more suggested structured quality indicators upon mapping at least a portion of the key words to the required categories of the quality indicator framework.

In some example embodiments, the query language is SQL or JavaScript.

In some example embodiments, the first database and the second database are linked, wherein the first database stores the selected structured quality indicator, and wherein the second database stores the unstructured quality indicator in association with the corresponding selected structured quality indicator.

In some example embodiments, the parsing of the unstructured quality indicator to identify key words includes identifying, using a library comprising one or more dictionaries, synonyms or corresponding official terminology for one or more words in the unstructured quality indicator.

In some example embodiments, a healthcare quality measurement system comprises: a user interaction module operable to receive a first unstructured quality indicator from a first healthcare quality delivery system; a natural language processing (NLP) engine including a quality indicator framework and a library, operable to parse the unstructured quality indicator to identify key words and relationships therebetween; a structured quality indicator generator operable to generate suggested structured quality indicators from unstructured quality indicators, the suggested structured quality indicators being standardized according to a predetermined standard; a quality measure engine operable to generate a query corresponding to a selected one of the structured quality indicators; a data repository operable to output result data from executing the query thereon; a quality measure dashboard operable to output the result data to the first healthcare quality delivery system.

In some example embodiments, the predetermined standard is Health Quality Measures Format (HQMF) or Health Level Seven (HL7).

In some example embodiments, the quality indicator framework includes categories onto which the identified keywords are mapped, the categories including required categories and optional categories, and wherein the structured quality indicator generator is further operable to generate the suggested structured quality indicators upon mapping at least a portion of the key words to the required categories of the quality indicator framework.

In some example embodiments, the query language is SQL or JavaScript.

In some example embodiments, a first database and a second database are linked to one another. The first database stores the selected structured quality indicator, and the second database stores the unstructured quality indicator in association with the corresponding selected structured quality indicator.

In some example embodiments, the parsing of the unstructured quality indicator to identify key words includes identifying, using the library comprising one or more dictionaries, synonyms or corresponding official terminology for one or more words in the unstructured quality indicator.

### BRIEF DESCRIPTION OF THE DRAWINGS

The present application will be more fully understood from the following detailed description taken in conjunction with the accompanying drawings, in which:

FIG. 1 illustrates a quality measurement environment, according to an exemplary embodiment;

FIG. 2 illustrates the quality measurement system of the quality measurement environment of FIG. 1;

FIG. 3 is a flowchart illustrating a process of obtaining quality measurement results, according to an exemplary embodiment;

FIG. 4 illustrates the quality indicator framework of the quality measurement environment of FIG. 1, according to an exemplary embodiment;

FIG. 5 is a Venn diagram illustrating the relationships between constituent parts of a quality indicator, according to an exemplary embodiment;

FIG. 6 is a Venn diagram illustrating suggested structured quality indicators, according to an exemplary embodiment; and

FIG. 7 illustrates a hospital dashboard for visualizing healthcare quality measurement results.

### DETAILED DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of various embodiments generally have similar features when those components are of a similar nature and/or serve a similar purpose.

The example embodiments presented herein are directed to systems and methods for measuring healthcare quality by dynamically specifying quality indicators and providing structured quality indicators. More specifically, an end-user such as a member of a healthcare delivery entity or organization inputs an unstructured quality indicator as free form text, speech-to-text, or the like. The unstructured quality indicator is parsed by a natural language processing (NLP) engine, thereby identifying the language, key words, relationships and other information regarding the unstructured quality indicator. The NLP engine uses a quality indicator framework to map the identified constituent parts of the unstructured quality indicator to categories of the framework. Based on this mapping, the NLP engine provides one or more suggestions for structured quality indicators. The suggested structured quality indicators are standardized or formatted quality indicators that most closely resemble the derived intended meaning of the user's input unstructured quality indicator. Once a user selects one of the suggested structured quality indicators, a quality measure engine generates a corresponding query language that is structured and in a query language specific to the data repository from which data is to be extracted. The quality measure engine executes the query on the data repository. The data returned form executing the query represents the healthcare quality results that are, in turn, rendered or caused to be rendered by a quality measurement dashboard accessible by the end-user.

### System

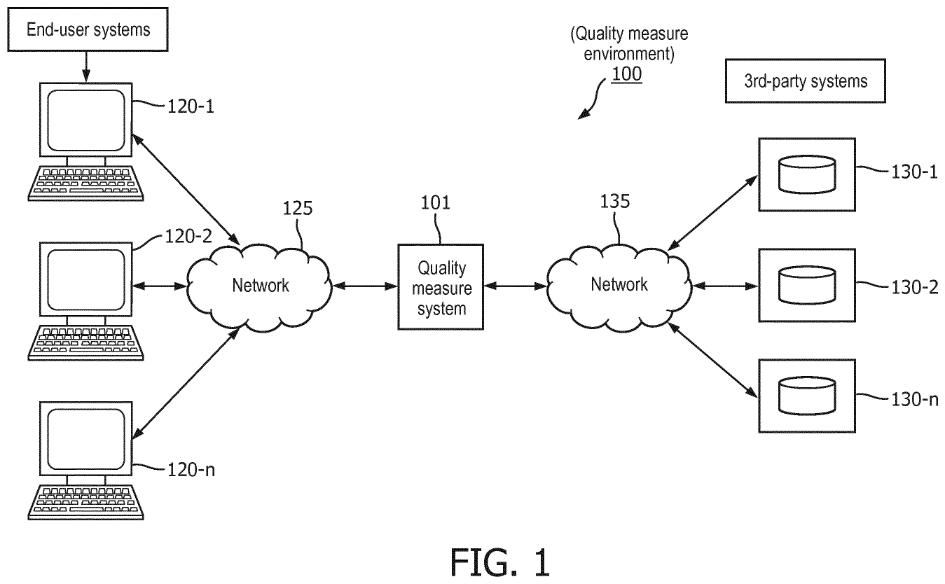

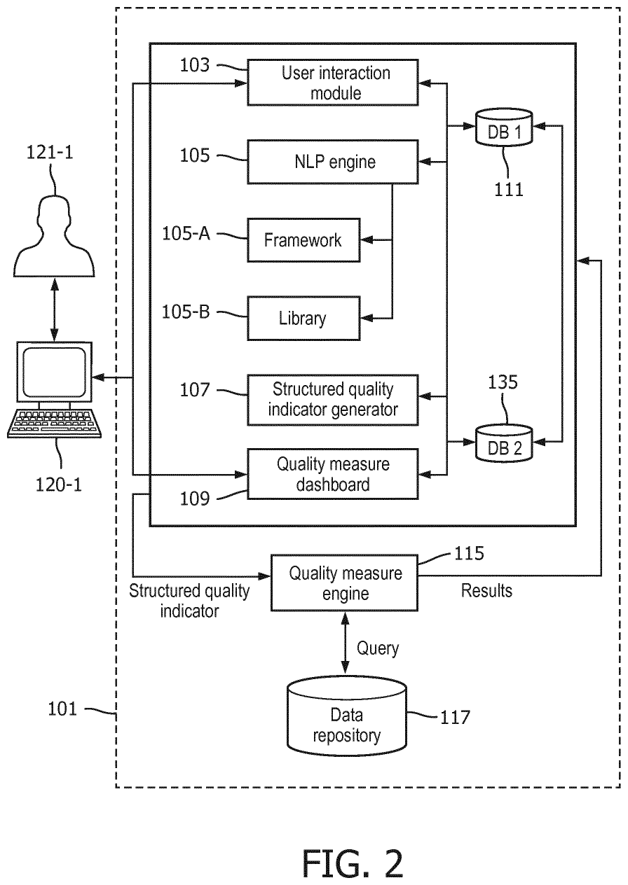

FIGS. 1 and 2 illustrate one exemplary embodiment of a quality measurement environment 100, including a quality measurement system 101, for measuring the quality of healthcare. As shown, the quality measurement environment 100 includes end-user systems 120-1, 120-2, . . . , and 120-n (collectively “120” or “end-user systems 120”) that are communicatively coupled to the quality measurement system 101 via a network 125. Some non-limiting examples of networks that can be used for communications between the end-user systems 120 and the quality measurement system 101 include a local area network (LAN), personal area network (PAN), wide area network (WAN), and the like.

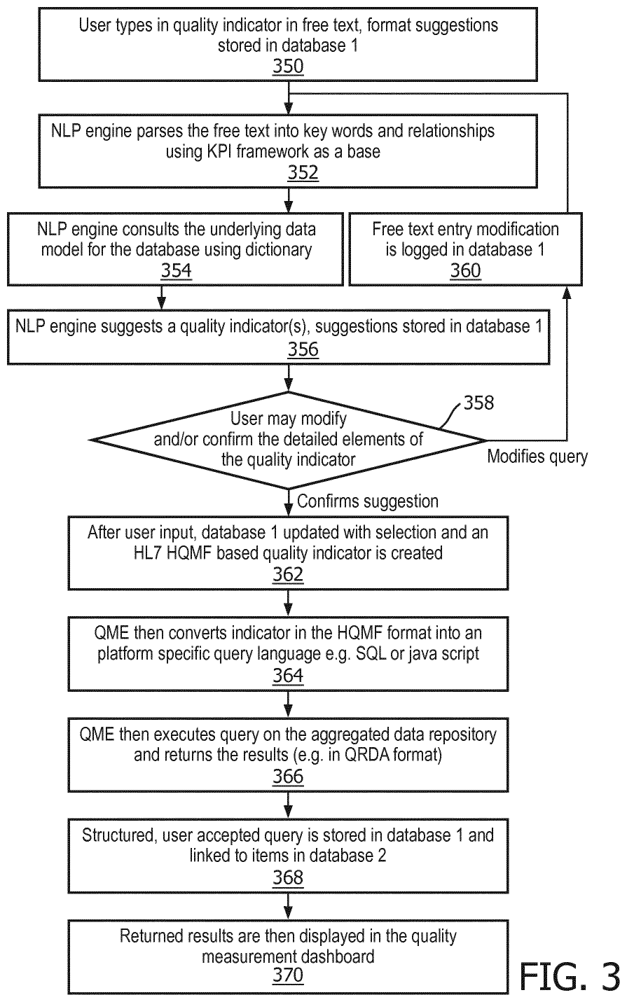

The end-user systems 120 are computing devices operated by end-users to obtain healthcare quality measure information. Some non-limiting examples of end-user systems 120 include personal computers, laptops, mobile devices, tablets and the like. Although not illustrated in FIG. 1, the end-user systems 120 can have or be associated with input/output devices, including monitors, projectors, speakers, microphones, keyboards, and the like. In some example embodiments, the users of the end-user systems 120 include data analysts, quality analysts, and/or C-level members (e.g., chief executive officer (CEO), chief marketing officer (CMO)), executives, and other care management staff of healthcare delivery entities (also referred to as healthcare delivery organizations). As described in further detail below with reference to step 350 of FIG. 3, the users of the end-user systems 120 input unstructured information such as a free text or speech quality indicator that is, in turn, processed to generate a structured quality indicator and query. Inputting the unstructured quality indicator can be done using the input/output devices of the end-user systems 120, such as the keyboard or microphone.

As described in further detail below with reference to FIG. 3, the quality measurement system 101 receives input unstructured information from the end-user systems 120 and processes it to generate quality measurement results. The generated quality measurement results can be output by the quality measurement system 101 to one or more of the end-user systems 120.

As shown, the quality measurement system 101 is also communicatively coupled to one or more third party systems 130-1, 130-2, . . . , and 130-n (collectively “130” or “third party systems 130”). The third party systems 130 may be and/or include databases that include information used to update or populate databases, libraries, and the like of the quality measurement system 101. For example, a third party system 130-1 can include a synonyms database that is used by the quality measurement system 101 to populate terms in one of its stored libraries, such as library 105-B shown in FIG. 2, which is described in further detail below.

As shown in FIG. 2, the quality measurement system 101 includes a data repository 117, a quality measure engine 115, and various components including a user interaction module 103, a natural language processer (NLP) engine 105, a quality indicator framework 105-A, the library 105-B, a structured quality indicator generator 107, a quality measure dashboard 109, a first database 111 and a second database 113.

The user interaction module 103 is a component of the quality measurement system 101, in the form of hardware and/or software, that is used to communicate with end-user systems such as the end-user system 120-1. As shown, the end-user system 120-1 is associated with an operator 121-1 such as an executive member of a healthcare delivery entity. In some embodiments, the user interaction module 103 provides a user interface for access by the end-user systems 120. The end-users, via their respective end-user systems 120, can input unstructured quality indicators via free text, recorded speech, or the like.

In turn, the input unstructured quality indicator is processed by the NLP engine 105 and the structured quality indicator generator 107 to produce a structured quality indicator. The quality measure engine 115 uses the structured quality indicator to generate a corresponding query and executes the query on the data repository 117. The data repository 117 outputs results from executing the query. It should be understood that the results may be or include various forms of data corresponding to the input unstructured quality indicator, the data being representable in multiple objects or formats.

The quality measure dashboard 109 renders or causes the rendering, at an end-user system 120, of the data resulting from the execution of the query. The quality measure dashboard 109 can render or cause to render the results in a variety of formats. The quality measure dashboard 109 thus enables end users such as members, staff, officers and/or directors of a quality healthcare delivery entity to intuitively visualize the requested data. Moreover, the dashboard 109 allows for real-time information resulting from executing the query to be monitored, allowing for more proactive responses to events and trends. It should be understood that although the dashboard 109 can be used to render or cause to render the result data, in some example embodiments, result data can be provided to end users in the form of reports, messages, or the like.

### Process

FIG. 3 illustrates a flowchart 300 for obtaining quality measurement results according to an exemplary embodiment. The quality measurement results can be generated using the quality measurement system 101. More specifically, quality measurement results are obtained by generating quality indicators and executing those against a data repository. Quality indicators are quantitative tools that are used to assess the clinical efficacy and performance of healthcare delivery entity or individual. Quality indicators are designed to determine whether the appropriate care has been provided given a set of clinical criteria and an evidence base. In some example embodiments, quality indicators are developed or endorsed by organizations such as the National Quality Forum (NQF). Table 1 below illustrates non-limiting examples of quality indicators, their type, and their corresponding operationalization. It should be understood that operationalization refers to a fuzzy concept that can be measured or observed using at least the listed quality indicators.

TABLE 1

| Indicator Type | Quality Indicator | Operationalization |
|---|---|---|
| Clinical | Mortality rates | Hospital mortality |
| | | Postoperative mortality (disease |
| | | specific) |
| | Patient satisfaction | Patient-reported outcome measures |
| | | (PROMs) |
| | | Quality-Adjusted Life Years |
| | | (QALYs) |
| | | Patient-reported experience |
| | | measures (PREMs) |
| | Hospital | Postoperative sepsis |
| | complications | Bed sores |
| | | Transfusion reactions |
| Operational | Waiting times | During admission/discharge/triage/ |
| | | diagnosis |
| | Length of stay | Length of stay in intensive care |
| | | unit (ICU) care |
| | | Length of stay at ward |
| | Asset utilization rate | Bed utilization rate |
| Financial | Hospital | Clinical cost reimbursement |
| | performance | |
| | Payer performance | % claims paid |
| | Physician | Revenue per physician |
| | performance | |

As shown in FIG. 3, at step 350, an end user enters unstructured data into a user interface made accessible by the user interaction module 103. The user interface can be configured to accept the input of unstructured data in various forms, including in typed free-form text, or in text entered by speaking and then converted to text using speech-to-text technology known by those skilled in the art. In one example embodiment, the input unstructured data is an unstructured quality indicator such as: “percentage of women over 40 who had a mammography.” The input unstructured quality indicator is stored in Database 2 (database 113). As discussed below in further detail with reference to step 368, entries of input unstructured quality indicators stored in the Database 2 (database 113) are linked to the suggested structured quality indicators and/or the selected one of the suggested structured quality indicators. Such linking allows Database 2 (database 112) to track user inputs and corresponding suggestions and selections, such that a set of learning rules can be developed.

It should be understood that the quality measurement system 101, including the user interaction module 103, can provide, via the user interface, auto-complete feedback during or upon completing the input of the unstructured quality indicator into the user interface. Data used to generate and provide the auto-complete feedback options is stored in an associated and accessible database, such as Database 1 (database 111) illustrated in FIG. 2. Database 1 (database 111) includes and or stores structured quality indicators, such as structured quality indicators previously accepted and/or approved by end-users.

In one example embodiment, the auto-complete feedback options are presented during the input of the unstructured quality indicator by the user 121-1 of the end-user system 120-1. The auto-complete feedback options can be, for example, suggested structured quality indicators that begin or include the part of the unstructured quality indicator input by the user 121-1 at the time of generating or presenting the auto-complete feedback options. The auto-complete feedback options can be displayed or caused to be displayed by the user-interaction module 103 at the user interface of the end-user system 120-1. The auto-complete feedback options can be displayed such that the part of the unstructured quality indicator that has been input is highlighted (e.g., color, bold text, etc.) or otherwise rendered in a manner that distinguishes it from the rest of the suggested structured quality indicators. The user 121-1 can continue to input the unstructured quality indicator using the text or speech functions, or can complete the input unstructured quality indicator by selecting one of the auto-complete feedback options.

At step 352, the NLP engine 105 parses the input unstructured quality indicator into key words or clauses, and derives their meanings and relationships, in order to identify the contextual constituent parts (or elements) of the unstructured quality indicator. In some example embodiments, words or clauses of the unstructured quality indicator are analyzed to identify an adjective or adjective clause, which can trigger the identification of a noun or noun clause that the adjective or adjective clause describes. This can be achieved using natural language processing algorithms understood by those skilled in the art. Understanding the meaning and relationships of the terms and clauses in the unstructured quality indicator enables the NLP engine 105 to more accurately and efficiently recognize or estimate the constituents parts (or elements) of the unstructured quality indicator and their intended meanings.

Once the meaning and relationships of the terms and clauses in the unstructured quality indicator have been analyzed and/or are further understood by the quality measurement system 101, the NLP engine 105 uses a quality indicator framework 105-A to map those identified terms and clauses to categories of the framework 105-A. In other words, the quality indicator framework 105-A, among other things, translates an unstructured, lay-man language indicator into a structured data format. The quality indicator framework 105-A also enables the identification of missing or needed information in order to accurately generate a structured quality indicator.

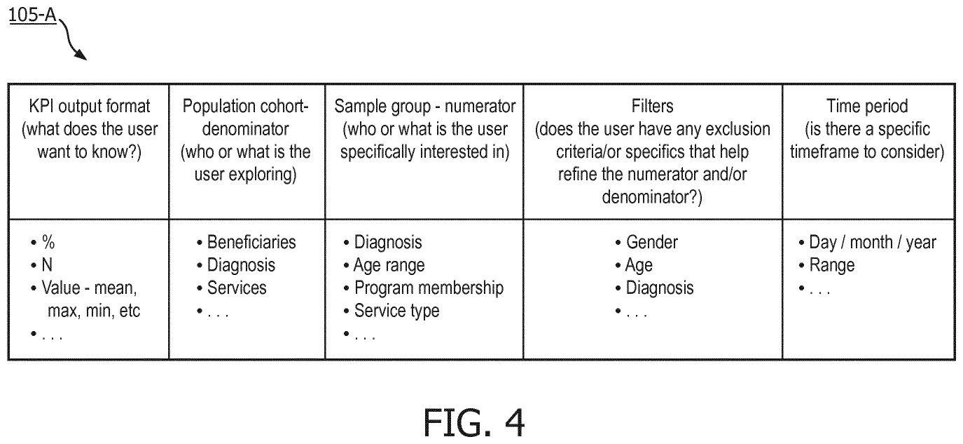

An exemplary quality indicator framework is illustrated in FIG. 4. As shown, the quality indicator includes framework categories, and criteria associated with each category. A non-exhaustive list of categories of the quality indicator framework 105-A can include:
Quality indicator output format (e.g., percentage, N, value)
Population cohort (denominator) (e.g., diagnosis, services)
Sample group (numerator) (e.g., diagnosis, age range)
Filters (e.g., gender, age)
Time period (e.g., day/month/year, range)

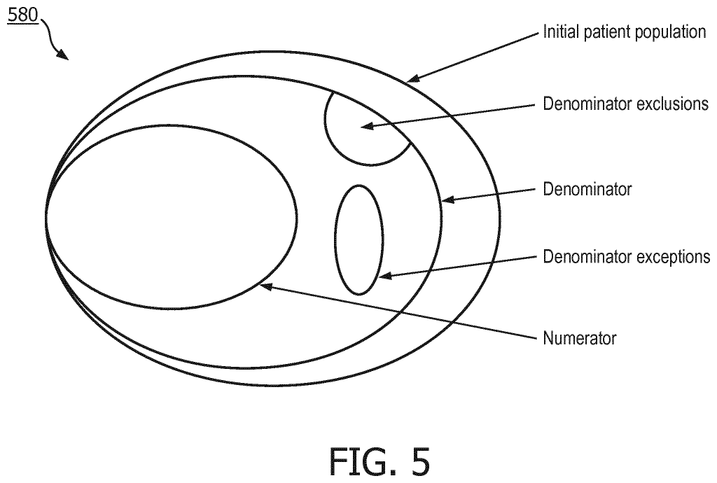

The categories of the quality indicator framework 105-a correspond to constituent parts of a structured quality indicator. FIG. 5 is a Venn diagram 580 illustrating the relationships between categories or constituent parts that are used to define the criteria for selecting the population for which quality information is sought. That is, the structured quality indicators provided herein enable the execution of corresponding queries for specific patient populations or cohorts.

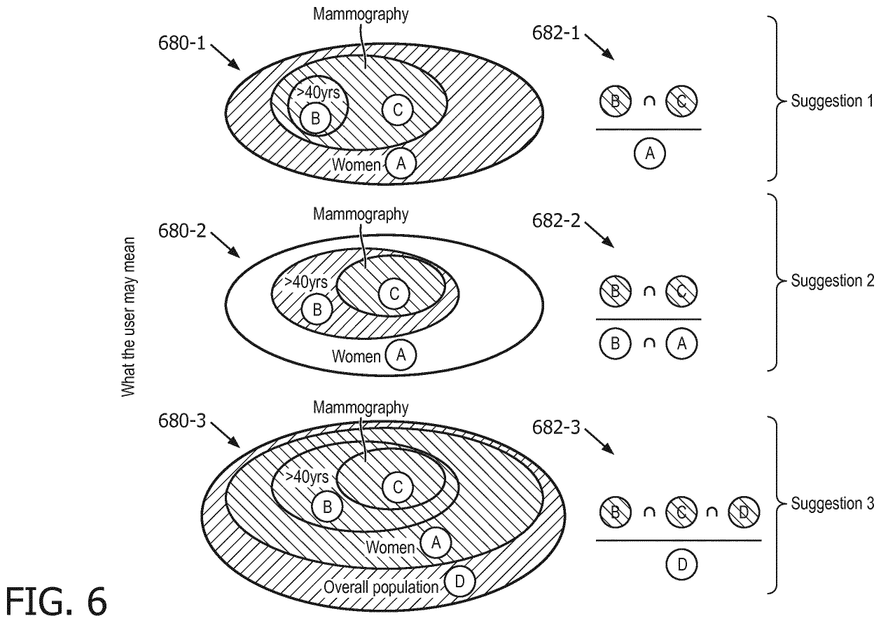

For instance, the Venn diagram 580 illustrates how the initial patient population (e.g., the patient population for which data is maintained in the data repository 117) is further narrowed or restricted by a nominator, denominator, exclusion or exception. Specific examples of Venn diagrams along the lines of Venn diagram 580 are described in further detail below with reference to FIG. 6. It should be understood that defining the criteria for selecting the population for which quality information is sought enables a structured quality indicator to be identified, and a corresponding query to be generated for execution against the data repository 117.

Still with reference to FIG. 4, the NLP engine 105 analyzes the criteria associated with each category of the quality indicator framework 105-A to determine if any of the terms or clauses identified from the parsing of the input unstructured quality indicator match or correspond to the criteria. In one example embodiment in which the end-user 121-1 inputs “percentage of women over 40 who had a mammography” as the unstructured quality indicator, the NLP engine 105 searches within the quality indicator framework 105-A to determine if any of the input terms or clauses can correspond to the criteria in the categories of the framework. For example, the NLP engine 105 can determine that the term “percentage,” from the unstructured quality indicator, can refer to a criteria within the “quality indicator output format” category; that the term “over 40” can refer to an age range within the “sample group (numerator)” category or the age criteria within the “filters” category; that “women” can refer to a gender criteria within the “filter” category; and that “had a mammography” can refer to a service criteria within the “population cohort (denominator)” category of the framework 105-A.

In some example embodiments, the quality indicator framework 105-A includes categories that are required, an others that are optional, to generate a structured quality indicator. For example, the quality indicator framework 105-A includes a category “time period,” which is used to limit the information sought to a certain date range, time period, or the like.

In some example embodiments, the quality indicator framework 105-A can include default values for categories. Such default values can be used by the NLP engine 105 in cases where the input unstructured quality indicator is missing information or if a value for a required constituent part is not initially identifiable from the parsed unstructured quality indicator. For example, if it is not clear what or who the user is exploring (i.e., the value for the “population cohort (denominator)” category from the unstructured quality indicator) from the parsing and mapping functions performed by the NLP engine 105, the quality indicator framework can default the value of the “population cohort (denominator)” to “overall population,” thereby indicating that the entire population, and not a subset thereof, is to be used as the target group.

Still with reference to FIG. 3, after the NLP engine 105, at step 352, parses the unstructured quality indicator and maps (or attempts to map) its words and clauses to categories of the quality indicator framework 105-A. The NLP engine 105, at step 354, uses the library 105-B to further analyze and/or obtain information about the words and/or clauses of the unstructured quality indicator. That is, the NLP engine 105 can use the library 105-B to identify potential or alternate meanings for terms in the unstructured quality indicator.

The library 105-B includes one or more dictionaries (or thesauruses). One type of dictionary may be a traditional dictionary and/or thesaurus that can identify and provide synonyms for terms in the input unstructured quality indicator. The NLP engine 105 can use such a dictionary, for example, to map terms or clauses in the unstructured quality indicator to categories in the quality indicator framework 105-A. For instance, in the example input quality indicator discussed above (“percentage of women over 40 who had a mammography”), if the NLP engine 105 is unable to map the term “mammography” to a category of the quality indicator framework 105-A, the NLP engine 105 can use the dictionary of the library 105-B to either obtain a synonym for mammography or to identify mammography as a type of medical imaging of a woman's breast. The NLP engine 105 can use the information obtained from the dictionary to, in turn, successfully map the term “mammography” to a category of the framework 105-A (e.g., population cohort (denominator) (e.g., services)).

Another type of dictionary that can be included in the library 105-B is a dictionary of official health problem and disease classifications and terminology, such as the International Statistical Classification of Diseases and Related Health Problems, 10th Revision (ICD-10) or International Classification of Primary Care, Second Edition (ICPC-2). An exemplary use of these types of dictionaries is described below with reference to step 356 of FIG. 3.

It should be understood that the dictionaries described above can be provided as separate dictionaries or can be compiled as a single dictionary within the library 105-B. Moreover, the dictionaries can be changed, updated or populated in the library 105-B using information obtained from third-party systems (e.g., third party systems 130), such as a database maintained by the United States' National Center for Health Statistics (NCHS), World Health Organization (WHO).

At step 356, the NLP engine 105 identifies suggested structured quality indicators based on the unstructured quality indicator input by the end user 121-1. The suggested structured quality indicators are those structured quality indicators identified or estimated by the NLP engine 105 as most closely matching the end-user's request for information. Identifying the closest structured quality indicators, which are in turn suggested by the NLP engine 105, is performed based on one or more of the parsing of the input unstructured quality indicator (step 352), the mapping of the parsed terms and clauses into categories of the quality indicator framework 105-A (step 352), and the searching for meanings and synonyms using the library 105-B to understand the meaning of terms or clauses in the unstructured quality indicator (step 354).

The suggested structured quality indicators are identified by the NLP engine 105 from among a set of structured quality indicators. The set of structured quality indicators can be stored, for example, in Database 1 (database 111). Although, it should be understood that structured quality indicators can be stored in other databases or systems that are accessible by the system 101. In some example embodiments, structured quality indicators can be obtained from and/or endorsed by third party systems 130, such as third party systems operated by organizations or entities that develop or endorse standardized quality indicators.

In some example embodiments, the stored structured quality indicators are structured such that they conform to a standard format. Moreover, the stored structured quality indicators can be pre-vetted, pre-endorsed, or pre-approved structured quality indicators. For example, such structured quality indicators may have been previously accepted by end-users. Or, such structured quality indicators may have been previously developed and/or endorsed by an organization such as the National Quality Forum (NQF). In some example embodiments, the structured quality indicators are stored such that they conform to a standard format, such as the NQF's Health Level Seven (HL7) standard known as the Health Quality Measures Format (HQMF). It should be understood that the stored structured quality indicators can be stored in various formats, including using the exemplary standards described above. It should also be understood that quality indicators can be measured as proportions, counts or values (e.g., average age of women undergoing a mammography; number of women undergoing a mammography in 2013).

Still with reference to step 356, the suggested structured quality indicators can be displayed in various formats, via a user interface of the system 120-1. For example, the suggested structured quality indicators can be listed in text form, graphically illustrated, or both. As shown in FIG. 6, in one example embodiment, suggested structured quality indicators can be illustrated by Venn diagrams and corresponding set notations. FIG. 6 illustrates examples of suggested structured quality indicators: Suggestion 1, Suggestion 2, and Suggestion 3. Each of the three suggestions (Suggestion 1, Suggestion 2, Suggestion 3) is illustrated in FIG. 6 with a Venn diagram (680-1, 680-2, and 680-3, respectively) and a corresponding notation (682-1, 682-2, 682-3, respectively).

As described above, the suggested structured quality indicators are identified based on the information derived from the input unstructured quality indicator in steps 352 and 354. That is, the NLP engine 105 uses derived information about the unstructured quality indicator to identify constituent parts and, in turn, uses the constituent parts to identify suggested structured quality indicators. In one example embodiment, the NLP engine 105 searches the stored structured quality indicators (e.g., in database 111) to identify matching instances of constituent parts of the input unstructured quality indicator that may be useful for the selection of the relevant population. In the case of an input unstructured quality indicator of: “percentage of women over 40 who had a mammography,” the NLP engine 105 searches the structured quality indicators stored in Database 1 (database 111) and determines that the identified constituent parts of “women” (A), “over 40” (B), and “had a mammography” (C) are found in three of the stored structured quality indicators, thus yielding the three suggested structured quality indicators shown in FIG. 6. In turn, the suggested structured quality indicators are output or caused to be displayed by the user interaction module 103, for example, via the user interface of the end-user system 120-1, such that the end-user can view and provide feedback (e.g., confirm, modify) regarding the suggestions.

It should be understood that, in some cases, suggested structured quality indicators can be represented differently (e.g., different Venn diagrams, set notations) but produce the same set of resulting data.

At step 358, the end-user 121-1 can confirm one of the suggested structured quality indicators as the desired structured quality indicator, or can modify one of the suggested structured quality indicators. The end-user's confirmation or modification are input via a user interface or input device of the end user system 120-1, and the input is in turn transmitted to the user interaction module 103 of the system 101. Confirming one of the suggested structured quality indicators causes the corresponding formatted structured quality indicator to be created, as described in further detail below with reference to step 362.

On the other hand, modifying one of the structured quality indicators causes the system to receive the user's modification and identify new or additional suggested structured quality indicators. More specifically, if the end user at step 358 does not confirm or accept any of the suggested structured quality indicators, the end user can instead input a modification to one of the suggestions in the same typed free text or speech-to-text manners, for example, described above with reference to step 350. In some example embodiments, modifications can be entered by manipulating a graphical illustration or notation associated with a suggested structured quality indicator. That is, a user can drag, drop, or perform other functions on or interaction with a Venn diagram or its set notation when displayed in the user interface of the end user system 120-1.

In some example embodiments, user modifications can include replacing, modifying or adding a word or words in or to one of the suggested structured quality indicators. The user's modification can be a result, for example, of the user determining that the NLP engine did not provide sufficiently accurate suggested structured quality indicators, or that omissions or mistakes existed in the input unstructured quality indicator. For example, in the case of the input unstructured quality indicator being “percentage of women over 40 who had a mammography,” the user can change “over 40” to “40 or older,” for instance, upon noticing that the initially input unstructured quality indicator did not encompass 40 year olds. The user may also add terms if it appears that the suggested structured quality indicators did not yield the user's desired denominator population (i.e., who the user is exploring), such as adding “Hispanic” to generate a new unstructured quality indicator (“percentage of Hispanic women over 40 who had a mammography”).

In the event that a user modifies one of the suggested structured quality indicators, the resulting modified quality indicator is treated as a new unstructured quality indicator. The modification and/or new unstructured quality indicator is/are stored or logged, at step 360 in Database 1 (database 111). In turn, the NLP engine 105 performs another iteration of steps 352, 354, 356 and 358 using the new unstructured quality indicator. That is, the new unstructured quality indicator is used to identify and provide new or additional suggested structured quality indicators (step 356) to the user 121-1. The user can then confirm one of the new or additional suggested structured quality indicators, or can modify one of those new or additional structured quality indicators, thereby proceeding to step 360.

In turn, once a user confirms or approves one of the suggested structured quality indicators at step 358—either based on initial suggestions or on subsequent suggestions after a user-modification of the quality indicator—the Database 1 (database 111) is updated at step 362 to include the selected structured quality indicator from among the suggestions. In this way, the Database 1 (database 111) can continuously be updated to accurately reflect or indicate which structured quality indicators have previously been approved or accepted by users.

Moreover, at step 362, the structured quality indicator generator 109 creates a formatted and/or standardized quality indicator based on the selected structured quality indicator from among the suggestions provided by the NLP engine 105. In some example embodiments, the structured quality indicator is formatted in accordance with HL7 HQMF, which represents the structured quality indicator as an electronic Extensible Markup Language (XML) document, such that the corresponding structured quality indicator can be enable or facilitate the automated creation of queries against the data repository 117. It should be understood that the structured quality indicator generated at step 362 may be formatted is accordance with any standards known by those skilled in the art, preferably in a manner that allows them to be used to generate queries compatible with electronic health records (EHRs) or other data repositories (e.g., health data repositories).

At step 364, the quality measure engine 115 converts the formatted structured quality indicator into an executable query. The quality measure engine 115 generates the query based on the specifications of the data repository 117 with which it is communicatively coupled. For example, the query is generated using a platform specific query language such as SQL or JavaScript that is executable and/or interpretable by the data repository 117. Thus, the quality measure engine 115 has access to information regarding the data and data model of the data repository 117. The executable query is designed to retrieve, from the data repository 117, the data required to fulfill the end user's initial request—i.e., the unstructured quality indicator.

In turn, at step 366, the quality measure engine 115 executes the query generated at step 364 against the data repository 117. Executing the query causes data needed to fulfill the end-user's request to be returned from the data repository 117 to the quality measure engine 115. The retrieved data can be returned in a response message of the same or different language as the query. In some example embodiments, the quality measure engine 115 executes multiple queries against multiple data repositories and combines the resulting data, for example, in scenarios in which data needed to fulfill the user's request cannot be found in a single source.

Moreover, at step 366, the data returned from the data repository 117 is arranged into a format that can be transmitted, and interpreted or processed by the system 101. For example, the data can be arranged in a Quality Reporting Document Architecture (QRDA) format, or the like.

At step 368, the query generated at step 364 is stored in or added to Database 1 (database 111). The entries of the query in Database 1 (database 111) are linked to entries in Database 2 (database 113). Database 2 (database 113) includes records or entries of input unstructured quality indicators and/or respective suggested structured quality indicators. Thus, linking the entries as discussed in step 368 causes each formatted query stored in the Database 1 (database 111) to be linked to (1) the user's input unstructured quality indicator from which the respective query was derived, and (2) the suggested quality indicators resulting from the input unstructured quality indicator.

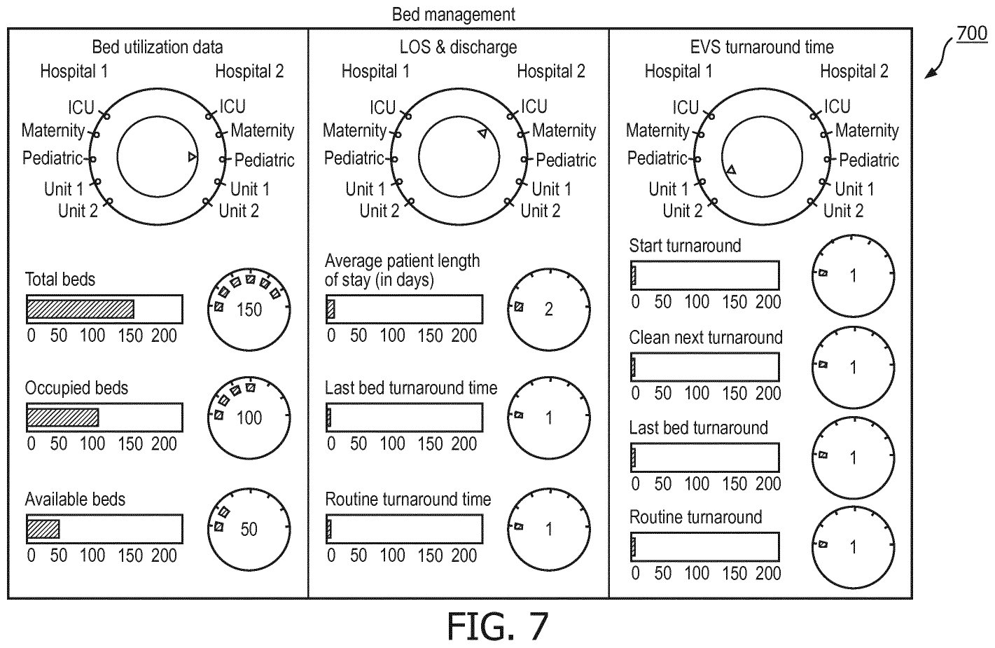

At step 370, the results returned from executing the query in step 366 are transmitted to the system 101. The quality measure dashboard 109 of the system 101 displays or causes to display, or renders or causes to render the results at or by the end-user system 120-1. The results displayed via the quality measure dashboard 109 can be presented in various formats, which can be customized according to an administrator of the system 101 or the end user 121-1. An example of a quality measure dashboard is illustrated in FIG. 7.

More specifically, FIG. 7 illustrates a hospital dashboard 700. As shown, the dashboard can display various data associated with the quality indicators of bed utilization rate, length of stay and discharge (“LOS & Discharge”), and hospital environmental services turnaround (“EVS Turnaround”). The dashboard allows the end-user to select the desired relevant population for each quality indicator, such as unit 1 of the hospital, unit 2, ICU, etc. For example, the displayed data associated with each quality indicator can include total beds, occupied beds, available beds, average patient length of stay (in days), last bed turnaround time, routine turnaround time, and the like. Moreover, as also shown in FIG. 7, the data can be displayed using text, numbers or objects such as pie charts, bar graphs, gauges, and the like. It should be understood that the dashboard, and its elements, can be configured in any way as desired by a system administrator, a user, or as the system determines the data is best illustrated.

In some example embodiments, the result data represents a complete set of information corresponding to the structured quality indicator. The complete set of information can be obtained, for example, when the information needed to satisfy each constituent part of the quality indicator can be identified in the data repository. On the other hand, in some example embodiments, result data may be incomplete, for example, due to an incomplete structured quality indicator or due to relevant information not existing or not being identifiable in the data repository. When result data is incomplete, the data repository may output the partial result data corresponding to the available information along with an explanation about the missing data. Alternatively, the data repository may not output any data in some embodiments when information relevant to the structured quality indicator is incomplete or completely missing.

In some example embodiments, the system 101 includes a closeness or similarity matching table that enables suggestions on how to improve a stored quality indicator (e.g., stored in Database 1) when or as new data elements become available in the data repository.

The present embodiments described herein can be implemented using hardware, software, or a combination thereof, and can be implemented in one or more computing device, mobile device or other processing systems. To the extent that manipulations performed by the present invention were referred to in terms of human operation, no such capability of a human operator is necessary in any of the operations described herein which form part of the present invention. Rather, the operations described herein are machine operations. Useful machines for performing the operations of the present invention include computers, laptops, mobile phones, smartphones, personal digital assistants (PDAs) or similar devices.

The example embodiments described above, including the systems and procedures depicted in or discussed in connection with FIGS. 1-7, or any part or function thereof, may be implemented by using hardware, software or a combination of the two. The implementation may be in one or more computers or other processing systems. While manipulations performed by these example embodiments may have been referred to in terms commonly associated with mental operations performed by a human operator, no human operator is needed to perform any of the operations described herein. In other words, the operations may be completely implemented with machine operations. Useful machines for performing the operation of the example embodiments presented herein include general purpose digital computers or similar devices.

Portions of the example embodiments of the invention may be conveniently implemented by using a conventional general purpose computer, a specialized digital computer and/or a microprocessor programmed according to the teachings of the present disclosure, as is apparent to those skilled in the computer art. Appropriate software coding may readily be prepared by skilled programmers based on the teachings of the present disclosure.

Some embodiments may also be implemented by the preparation of application-specific integrated circuits, field programmable gate arrays, or by interconnecting an appropriate network of conventional component circuits.

Some embodiments include a computer program product. The computer program product may be a non-transitory storage medium or media having instructions stored thereon or therein which can be used to control, or cause, a computer to perform any of the procedures of the example embodiments of the invention. The storage medium may include without limitation a floppy disk, a mini disk, an optical disc, a Blu-ray Disc, a DVD, a CD or CD-ROM, a micro-drive, a magneto-optical disk, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the non-transitory computer readable medium or media, some implementations include software for controlling both the hardware of the general and/or special computer or microprocessor, and for enabling the computer or microprocessor to interact with a human user or other mechanism utilizing the results of the example embodiments of the invention. Such software may include without limitation device drivers, operating systems, and user applications. Ultimately, such computer readable media further includes software for performing example aspects of the invention, as described above.

Included in the programming and/or software of the general and/or special purpose computer or microprocessor are software modules for implementing the procedures described above.

While various example embodiments of the invention have been described above, it should be understood that they have been presented by way of example, and not limitation. It is apparent to persons skilled in the relevant art(s) that various changes in form and detail can be made therein. Thus, the disclosure should not be limited by any of the above described example embodiments, but should be defined only in accordance with the following claims and their equivalents.

In addition, it should be understood that the figures are presented for example purposes only. The architecture of the example embodiments presented herein is sufficiently flexible and configurable, such that it may be utilized and navigated in ways other than that shown in the accompanying figures.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that the procedures recited in the claims need not be performed in the order presented.

1. A healthcare quality measurement system comprising:
at least one memory operable to store a data repository, a first database and a second database;
a processor communicatively coupled to the at least one memory, the processor being operable to:
receive an unstructured quality indicator from one of a plurality of end-user systems;
parse the unstructured quality indicator to identify key words, and relationships therebetween;
map the key words to categories of a quality indicator framework, the categories of the quality indicator framework corresponding to one or more constituent parts of one or more candidate structured quality indicators;
identify one or more suggested structured quality indicators from among the one or more candidate structured quality indicators, based at least on the key words mapped to the categories of the quality indicator framework;
receive a selection of a structured quality indicator from among the one or more suggested structured quality indicators;
generate a query corresponding to the structured quality indicator, the query being generated in a query language executable on the data repository;
execute the query against the data repository to obtain result data, the results including information relating to the unstructured quality indicator and obtained based on the structured quality indicator; and
output the result data obtained by executing the query,
wherein the candidate structured quality indicators are quality indicators standardized according to a predetermined standard.

2. The system of claim 1, wherein the result data is output via a quality measurement dashboard.

3. The system of claim 1, wherein the predetermined standard is Health Quality Measures Format (HQMF) or Health Level Seven (HL7).

4. The system of claim 1,
wherein the categories of the quality indicator framework include required categories and optional categories, and
wherein the processor is operable to identify the one or more suggested structured quality indicators upon mapping at least a portion of the key words to the required categories of the quality indicator framework.

5. The system of claim 1, wherein the query language is SQL or JavaScript.

6. The system of claim 1,
wherein the first database and the second database are linked,
wherein the first database stores the selected structured quality indicator, and
wherein the second database stores the unstructured quality indicator in association with the corresponding selected structured quality indicator.

7. The system of claim 1,
wherein the parsing of the unstructured quality indicator to identify key words includes identifying, using a library comprising one or more dictionaries, synonyms or corresponding official terminology for one or more words in the unstructured quality indicator.

8. A method of providing healthcare quality measurements, comprising:
receiving an unstructured quality indicator from one of a plurality of end-user system;
parsing the unstructured quality indicator to identify key words, and relationships therebetween;
mapping the key words to categories of a quality indicator framework, the categories of the quality indicator framework corresponding to one or more constituent parts of one or more candidate structured quality indicators;
identifying one or more suggested structured quality indicators from among the one or more candidate quality indicators, based at least on the key words mapped to the categories of the quality indicator framework;
receiving a selection of a structured quality indicator from among the one or more suggested structured quality indicators;
generating a query corresponding to the structured quality indicator, the query being generated in a query language executable on a data repository;
executing the query against the data repository to obtain result data, the results including information relating to the unstructured quality indicator and obtained based on the structured quality indicator; and
outputting the result data obtained by executing the query,
wherein the candidate structured quality indicators are quality indicators standardized according to a predetermined standard.

9. The method of claim 8, wherein the result data is output via a quality measurement dashboard.

10. The method of claim 8, wherein the predetermined standard is Health Quality Measures Format (HQMF) or Health Level Seven (HL7).

11. The method of claim 8,
wherein the categories of the quality indicator framework include required categories and optional categories, and
wherein the method further comprises identifying the one or more suggested structured quality indicators upon mapping at least a portion of the key words to the required categories of the quality indicator framework.

12. The method of claim 8, wherein the query language is SQL or JavaScript.

13. The method of claim 8,
wherein the first database and the second database are linked,
wherein the first database stores the selected structured quality indicator, and
wherein the second database stores the unstructured quality indicator in association with the corresponding selected structured quality indicator.

14. The method of claim 8,
wherein the parsing of the unstructured quality indicator to identify key words includes identifying, using a library comprising one or more dictionaries, synonyms or corresponding official terminology for one or more words in the unstructured quality indicator.

15. A healthcare quality measurement system comprising:
a user interaction module operable to receive a first unstructured quality indicator from a first healthcare quality delivery system;
a natural language processing (NLP) engine including a quality indicator framework and a library, operable to parse the unstructured quality indicator to identify key words and relationships therebetween;
a structured quality indicator generator operable to generate suggested structured quality indicators from unstructured quality indicators, the suggested structured quality indicators being standardized according to a predetermined standard;
a quality measure engine operable to generate a query corresponding to a selected one of the structured quality indicators;
a data repository operable to output result data from executing the query thereon;
a quality measure dashboard operable to output the result data to the first healthcare quality delivery system.

16. The system of claim 15, wherein the predetermined standard is Health Quality Measures Format (HQMF) or Health Level Seven (HL7).

17. The system of claim 15,
wherein the quality indicator framework includes categories onto which the identified keywords are mapped, the categories including required categories and optional categories, and
wherein the structured quality indicator generator is further operable to generate the suggested structured quality indicators upon mapping at least a portion of the key words to the required categories of the quality indicator framework.

18. The system of claim 15, wherein the query language is SQL or JavaScript.

19. The system of claim 15, further comprising: a first database and a second database linked to one another,
wherein the first database stores the selected structured quality indicator, and
wherein the second database stores the unstructured quality indicator in association with the corresponding selected structured quality indicator.

20. The system of claim 15,
wherein the parsing of the unstructured quality indicator to identify key words includes identifying, using the library comprising one or more dictionaries, synonyms or corresponding official terminology for one or more words in the unstructured quality indicator.

21. A computer program product comprising a non-transitory storage medium or media having instructions stored thereon that, when executed by a computer, cause the computer to perform the method of claim 8.
